(19) United States
(12) Patent Application Publication
(10) Pub. No.: US 2005/0002878 A1
(43) Pub. Date: Jan. 6, 2005

(54) Use of tazarotene for preparing a nail varnish for treating and/or preventing psoriasis and nail varnish containing same

(76) Inventors: Pascal Lefrancois, Lavaur (FR); Roger Navarro, Pamiers (FR); Marlene Delaunois, Villefrance-DE Lauragais (FR)

Correspondence Address: BURNS DOANE SWECKER & MATHIS L L P, POST OFFICE BOX 1404, ALEXANDRIA, VA, 22313-1404, US

(21) Appl. No.: 10/491,666

(22) PCT Filed: Oct. 3, 2002
(86) PCT No.: PCT/FR02/03366

(30) Foreign Application Priority Data
Oct. 5, 2001 (FR) 01/12,825

(51) Int. Cl. A61K 7/04

(52) U.S. Cl. 424/61

(57) ABSTRACT

The invention concerns the use of tazarotene for preparing a nail varnish for treating and/or preventing psoriasis and nail varnish containing tazarotene and method for making same. More particularly, the invention concerns a nail varnish containing additionally to tazarotene a film-forming agent soluble or insoluble in polar solvents and physiologically acceptable solvents. Optionally it contains a penetration agent.

The present invention relates to the use of tazarotene for preparing a nail varnish for treating and/or preventing psoriasis, to a nail varnish containing tazarotene, and to a method of production.

Psoriasis is a skin disease which affects more than two percent of western populations.

It results in chronic erythematosquamous dermatosis. The lesion combines keratinocyte abnormalities (greatly increased mitotic activity and abnormal differentiation) with dermal and epidermal inflammatory phenomena.

The disease is controlled by genetic factors, which are revealed by various environmental factors.

Nail conditions due to psoriasis are persistent disease forms which it has not been possible, up until now, to treat satisfactorily. They affect, depending on the country, between 10 and 78% of populations developing psoriasis.

Today, the most widespread treatment for nail psoriasis consists of subcutaneous injections of corticoids.

These corticoids are anti-inflammatory active principles widely used for dermatological treatments.

These very painful injections can be accompanied by topical treatment.

Another treatment consists in applying locally to the nails specific substances with antipsoriatic action, in the form of a cream. In this field, the most diverse methods of treatment have been tried. Thus, in a combined treatment, the nails have first been treated with solutions of substances with antipsoriatic action and dressings have been applied with cream overnight. This method of treatment is also very unpleasant and psychologically trying for the patients. Firstly, it is necessary to treat the nails several times a day. Secondly, the nails must be wrapped in dressings, especially overnight. Consequently, the treatment, which usually lasts several months, is not commonly pursued by the patients, who, on the contrary, become discouraged and neglect the treatment. This results in the treatment failing. In this method, the success of the treatment is also compromised by the fact that the solutions and the creams are usually water-miscible or hydrophilic and can be removed from the surface of the nail or entrained away from the nail by dissolution while washing, taking a bath or having a shower, and must therefore then be applied again.

Tazarotene is a retinoid derivative known for its antipsoriatic activity.

It is a retinoid derivative, also known as ethyl 6-[(3,4-dihydro-4,4-dimethyl-2H-1-benthiopyran-6-yl)ethynyl]-3-pyridinecarboxylate, of formula

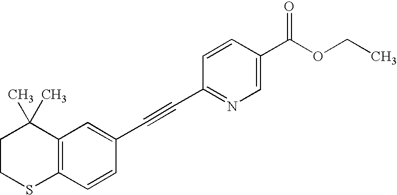

It may in particular be in the form of a cream or a gel.

However, while the effectiveness of tazarotene on plaques of psoriasis have been demonstrated, treating psoriasis which develops under the nails is delicate.

It is difficult to reach effective concentrations of active principle so as to eliminate the psoriasis under these conditions.

It has now been found that it is possible to treat nail psoriasis successfully and/or to prevent it by applying the nail varnish according to the invention to the nails, and in particular to the nails affected by psoriasis.

A subject of the present invention is therefore the use of tazarotene for preparing a nail varnish for treating and/or preventing psoriasis.

A second subject of the invention is a nail varnish with antipsoriatic action containing tazarotene.

The nail varnish according to the invention advantageously contains a film-forming agent which is soluble or insoluble in polar solvents.

The subject of the present invention is also a nail varnish containing, besides tazarotene and a film-forming agent which is soluble or insoluble in polar solvents,
a) one or more physiologically acceptable solvents suitable for the type of film-forming agent used,
b) optionally, one or more penetration agents and, optionally,
c) additives conventionally used in cosmetics.

According to the invention, the nail varnish should contain sufficient active principle so that, when it is deposited on the nail, this allows an appropriate release ensuring diffusion up to the nail bed, and thus making it possible to attain the therapeutic threshold.

Another subject of the present invention is a method for producing the nail varnish according to the invention, which comprises a step consisting in mixing a film-forming agent and tazarotene. Advantageously, when the nail varnish which is the subject of the present invention contains constituents a), b) and/or c), mentioned above, other than the active principle and the film-forming agent, the active principle and the other constituents are dissolved in the solvent(s) before the film-forming agent.

The nail varnishes according to the invention may contain film-forming agents which are soluble in aqueous-alcoholic solvents or nonpolar solvents which, after drying on the nail, form water-resistant films. Suitable film-forming agents which are soluble in nonpolar solvents (or insoluble in polar solvents) include substances based on cellulose nitrate or on synthetic polymers well known to the formulator for their innocuity. Mention may be made, for example, of poly(vinyl acrylate) and partially saponified poly(vinyl acrylate), copolymers of first vinyl acetate and secondly acrylic acid or crotonic acid or a monoalkyl maleate, ternary copolymers of firstly vinyl acetate and secondly crotonic acid and vinyl neodecanoate or crotonic acid and vinyl propionate, copolymers of vinyl methyl ether and of a monoalkyl maleate, in particular in the form of monobutyl maleate, copolymers of fatty acid vinyl esters and of acrylic acid or of methacrylic acid, copolymers of N-vinylpyrrolidone, methacrylic acid and alkyl methacrylate, copolymers of acrylic acid and of methacrylic acid, or of alkyl acrylate or of alkyl methacrylate, poly(vinyl acetals) and poly(vinyl butyrals), poly(N-vinylpyrrolidones) substituted with alkyl groups, alkyl esters of copolymers of olefin and of maleic anhydride and products from reaction of rosin and of acrylic acid. In the ester, the alkyl residues are usually short chains and generally contain no more than four carbon atoms.

Suitable film-forming agents which are soluble in polar solvents include polyacrylamides; acrylic/methacrylic, polymethacrylate/butyl acrylate and acrylic/acrylate copolymers; polyvinyl alcohol; poly(vinyl methyl ether)/maleic anhydride copolymer; polyvinyl-pyrrolidone; polyvinylpyrrolidone/vinyl acetate copolymer and vinylpyrrolidone/dimethylaminoethyl methacrylate copolymer.

A mixture of film-forming agents of the same class (soluble in polar solvents or else soluble in nonpolar solvents) can be used as film-forming agent in the context of the invention.

Among the film-forming agents which are soluble in polar solvents, preference is given to acrylic/acrylate and acrylic/methacrylate copolymers or the acrylic/acrylate/methacrylate tercopolymer.

Among the film-forming agents which are soluble in nonpolar solvents, preference is given to the vinyl methyl ether/monobutyl maleate copolymer.

Among the physiologically acceptable solvents, and as nonpolar solvents, mention may be made of substances such as hydrocarbons, halogenated hydrocarbons, alcohols, ethers, ketones and esters, commonly used in cosmetics, in particular esters of acetic acid and of monoalcohols, such as ethyl acetate and butyl acetate, optionally as a mixture with aromatic hydrocarbons such as toluene and/or alcohols such as ethanol or isopropyl alcohol. When a water-soluble film-forming agent is used as film-forming agent, use may be made, as solvent, in the context of the present invention, of alcohols such as ethanol, isopropyl alcohol or any other alcohol having a boiling point sufficiently low to have a very short drying time. These alcohols may be present in the varnish in the form of mixtures, and an amount of water ranging from 0 to 20% by weight relative to the total amount of solvent can then be added. These are solvents referred to as aqueous-alcoholic solvents.

In the context of the present invention, and whether the film-forming agent is water-soluble or -insoluble, use is preferably made of a combination of solvents or solvent systems. These solvents are predominantly important with respect to the drying time, the ease of application with a brush and other important properties of the varnish or of the film of varnish. The solvent system may be between 70 and 94% by weight relative to the total weight of the varnish.

This solvent system is preferably a mixture of solvents with a low boiling point (=boiling point up to 100° C.) and of solvents with a medium boiling point (=boiling point up to 150° C.), optionally with a small proportion of solvents with a high boiling point (=boiling point up to 200° C.).

As penetration agents, mention may in particular be made of urea, oleic acid and ethoxydiglycol. Any other penetration agent commonly used in this type of formulation may also be used in the context of the present invention.

The nail varnishes according to the invention may also contain additives commonly used in cosmetics, such as plasticizers based on phthalate, urea or camphor, colorants or colored pigments, a pearlescent agent, sedimentation retarders, sulfonamide resins, silicates, fragrances, wetting agents, such as sodium dioctyl-sulfosuccinate, lanolin derivatives, light-screening agents, such as 2-hydroxy-4-methoxybenbzophenone, substances with antibacterial action and substances with keratolytic and/or keratoplastic action, such as ammonium sulfite, esters and salts of thioglycolic acid, urea, allantoin, enzymes and salicylic acid.

Colored or pigmented nail varnishes offer, for example, the advantage that the composition according to the invention can be adapted to the patient's esthetic preferences.

The nail varnish is prepared in the usual manner, by mixing of the individual components and, if necessary, subsequent treatment adapted to each composition.

In the nail varnish according to the invention, the tazarotene content is generally an amount ranging from 0.1 to 4%, preferably from 0.1 to 2% by weight relative to the total weight of the composition.

The nail varnish according to the invention generally contains from 0.1 to 30%, preferably from 5 to 20% by weight of film-forming agent, from 69 to 99% by weight, preferably from 79 to 94%, and more precisely from 81 to 86% by weight of solvent, and from 0.5 to 15% by weight, preferably from 1 to 10% and more precisely from 3 to 7% of penetration agent relative to the total weight of the varnish.

The proportions mentioned above make it possible in particular to obtain a varnish suitable for the production chain while at the same time exhibiting properties of ease of spreading and rapid drying.

It is known that the superficial cornified layers have, inter alia, the biological function of preventing the penetration of foreign substances. The compositions according to the invention enable a considerable proportion of the active principle to pass through the 168 superficial cornified layers and thus to exert a long-lasting, deep action.

The present invention relates, moreover, as a particular embodiment, to a varnish as described above, also containing a corticoid or a mixture of various corticoids.

The corticoids which can be combined with the tazarotene can be chosen from the corticoids having:
very high activity, among which are clobetasol propionate and betamethasone dipropionate;
high activity, among which: betamethasone valerate or dipropionate, fluocinolone acetonide and hydrocortisone aceponate or butyrate;
quite high activity, among which: flucinolone acetonide, and desonide;
moderate activity, among which: hydrocortisone acetate.

As nail varnish containing a corticoid, a nail varnish containing clobetasol or one of its pharmaceutically acceptable salts, such as clobetasol propionate, is preferred.

This corticoid classification is the European classification and is not limiting for the invention. The amount of corticoid can range from 0.01% to 5% by weight relative to the total weight of varnish, as a function of the seriousness of the psoriasis to be treated.

The present invention is illustrated in greater detail using the following example.

### EXAMPLE:

Study of the Activity of 6 Formulations of Varnish According to the Invention

In the tests on penetration capacity, several formulae were tested, prepared either with film-forming agents tending toward being hydrophilic, dissolved in aqueous-alcoholic media, or with film-forming agents tending toward being hydrophobic, dissolved in relatively nonpolar solvents.

In the two cases, we concentrated on the non-solubilization in water, after drying, of the film formed, which would, when taking a wash, lead to the disappearance of the active principle and therefore to loss of the activity of the product.

A first series of tests was carried out on skin explants in order to verify, on this conventional model, whether we were able to discriminate several different formulae.

Using diffusion cells of the modified Franz cell type, the various formulae were applied in finite doses, i.e. 9.2 mg/cm², to human abdominal skin cut at 400 microns on a dermatome. Passage through was studied over 24 hours. The tazarotene active principle diffused through the skin and was collected in the recipient liquid at 3 h, 6 h, 12 h and 24 h.

After 24 h, the active principle was assayed in the tissue, at the end of the diffusion phase (dermis and epidermis) and at the surface of the tissue, after having surface cleaned the varnish.

Two types of formulae were tested:

| | | | | Apolar formulae | | | |
|---|---|---|---|---|---|---|---|
| | Polar formulae | | | (in %) | | | |
| | (in %) | | | | | | Gel |
| | 1 | 2 | 3 | 4 | 5 | 6 | control |
| TAZAROTENE | 0.5 | 0.5 | 0.1 | 1 | 0.5 | 0.1 | 0.1 |
| B.H.T. | 0.05 | 0.05 | 0.05 | 0.05 | 0.05 | 0.05 | — |
| UREA | — | 5 | — | — | — | — | — |
| COPOLYMER: | 14 | 14 | 14 | — | — | — | — |
| ACRYLIC ACID/ | | | | | | | |
| METHYL METHACRYLATE/ | | | | | | | |
| ETHYL ACRYLATE | | | | | | | |
| PURIFIED WATER | 9.99 | 9.41 | 10.04 | — | — | — | — |
| ETHANOL | 75.46 | 71.04 | 75.81 | — | — | — | — |
| COPOLYMER: | — | — | — | 15 | 15 | 15 | — |
| VINYL METHYL ETHER | | | | | | | |
| MONOBUTYL ESTER OF | | | | | | | |
| MALEIC ACID | | | | | | | |
| ISOPRANOL | — | — | — | 50.15 | 50.45 | 50.69 | — |
| ETHYL ACETATE | — | — | — | 33.80 | 34.00 | 34.16 | — |

B.H.T.: Butylated Hydroxytoluene

With polar film-forming agent in solvent (formulae 1, 2 and 3)

With nonpolar film-forming agent and solvent (formulae 5 3, 4 and 5)

We used a commercial control, known as Zorac®, a dermal gel containing a dose of 0.1% of tazarotene.

Study of the transcutaneous passage of tazarotene over 24 hours.

| | | Passage in |
|---|---|---|
| | Characteristic | micrograms |
| Formula 1 | Hydrophilic varnish | 0.175 |
| | 0.5% tazarotene | |
| | 5% urea | |
| Formula 2 | Lipophilic varnish | 0.12 |
| | 1% tazarotene | |
| Formula 3 | Lipophilic varnish | 0.065 |
| | 0.5% tazarotene | |
| Formula 4 | Hydrophilic varnish | 0.05 |
| | 0.5% tazarotene | |
| Formula 5 | 0.1% tazarotene gel | 0.012 |

Results: We Demonstrated:
that the higher the concentration of active principle, the greater the penetration of the active principle,
that whatever the solvent and the type of film-forming agent, the active principle at the same dose penetrates in the same manner,
that the addition of urea promotes trans-nail passage in the case of the polar-type formula.

A second series of tests for penetration was carried out on bovine horn.

These bovine horns were cut so as to obtain disks of keratin having a diameter compatible with that of the Franz cells, these disks having a standardized thickness of 0.6 mm.

The Franz cell used in this experiment is in the form of receptacle with two compartments separated by the skin or the horn. In the lower compartment is a solution of physiological saline thermostated at 37° C. The product to be studied is placed in the upper receptable, in contact with the skin or the horn. Regular samples are taken from the lower compartment, in order to study the amounts of active principle.

The varnish containing the tazarotene is deposited onto this disk of horn and the trans-nail passage is monitored using C14-labeled tazarotene. The method of studying the penetrating capacity on bovine horn makes it possible to study the compounds with regard to their penetration capacity at a concentration which is effective with respect to the horn, the composition of which is very similar to the composition of the nail.

7.1 mg/cm² of varnish as defined in formulae 1 to 6 are deposited on the bovine horn.

The contact time was 48 hours, with samples being taken at 6 h, 12 h, 24 h, 36 h and 48 h. The nail was cut in sections of 40 microns on a dermatome, the surface of the horn and the base of the horn, making up 240 microns.

Study of the trans-nail passage of tazarotene over 24 hours

| | | Passage in |
|---|---|---|
| | Characteristic | micrograms |
| Formula 1 | Hydrophilic varnish | 0.001 |
| | 0.5% tazarotene | |
| Formula 2 | Hydrophilic varnish | 0.05 |
| | 0.5% tazarotene | |
| | Urea | |
| Formula 3 | Lipophilic varnish | 2 |
| | 1% tazarotene | |
| Formula 4 | Lipophilic varnish | 0.025 |
| | 0.5% tazarotene | |

The results were the same. The product with polar solvent and which contains urea gives better trans-nail passage.

In conclusion: The nail varnishes containing a film-forming agent and a solvent according to the invention, whether they are based on polar solvents and on film-forming agents of the same type with or without urea, or whether they are based on nonpolar solvents, pass through the nail to an extent which makes it possible to attain the therapeutic threshold.

These varnishes make it possible to obtain rapid and painless effectiveness, unlike the other known therapies for this condition.

1. A method for treating psoriasis comprising applying to a nail surface or nail polish comprising an effective amount of tazarotene.

2. A nail varnish with antipsoriatic action comprising an effective amount of tazarotene.

3. The nail varnish with antipsoriatic action as claimed in claim 2, which further comprises a film-forming agent which is soluble or insoluble in polar solvents.

4. The nail varnish with antipsoriatic action as claimed in claim 3, which further comprises as a film-forming agent, one or more physiologically acceptable solvents suitable for the type of film-forming agent used.

5. The nail varnish with antipsoriatic action as claimed in claim 4, which comprise one or more penetration agents and, optionally, additives conventionally used in cosmetics.

6. The nail varnish as claimed in claim 5, characterized in that wherein the penetration agent is selected from urea, oleic acid and ethoxydiglycol.

7. The nail varnish as claimed in claim 2, which comprises tazarotene in an amount ranging from 0.1 to 4% by weight relative to the total weight of the composition.

8. The nail varnish as claimed in claim 7, comprising contains tazarotene in an amount ranging from 0.1% to 4% by weight relative to the total weight of the composition.

9. The nail varnish as claimed in claim 3, wherein the film-forming agent which is soluble in nonpolar solvents is selected from cellulose nitrate, poly(vinyl acrylate) and partially saponified poly(vinyl acrylate), copolymers of first vinyl acetate and secondly acrylic acid or crotonic acid or a monoalkyl maleate, ternary copolymers of firstly vinyl acetate and secondly crotonic acid and vinyl neodecanoate or crotonic acid and vinyl propionate, copolymers of vinyl methyl ether and of a monoalkyl maleate, copolymers of fatty acid vinyl esters and of acrylic acid or of methacrylic acid, copolymers of N-vinylpyrrolidone, methacrylic acid and alkyl methacrylate, copolymers of acrylic acid and of methacrylic acid, or of alkyl acrylate or of alkyl methacrylate, poly(vinyl acetals) and poly(vinyl butyrals), poly(N-vinylpyrrolidones) substituted with alkyl groups, alkyl esters of copolymers of olefin and of maleic anhydride and products from reaction of rosin and of acrylic acid.

10. The nail varnish as claimed in claim 3, wherein the film-forming agent which is soluble in polar solvents is selected from polyacrylamides; acrylic/methacrylic, polymethacrylate/butyl acrylate and acrylic/acrylate copolymers; polyvinyl alcohol; poly(vinyl methyl ether)/maleic anhydride copolymer; polyvinylpyrrolidone; polyvinylpyrrolidone/vinyl acetate copolymer and vinylpyrrolidone/dimethylaminoethyl methacrylate copolymer.

11. A method for preparing a the nail varnish as claimed in claim 3, comprising mixing a water-soluble or -insoluble film-forming agent with tazarotene and, optionally, with other usual components to prepare nail varnishes.

12. The nail varnish as claimed in claim 3, further comprising a corticoid or a mixture of various corticoids.

13. The nail varnish as claimed in claim 12, wherein the corticoid is selected from clobetasol propionate or betamethasone dipropionate, betamethasone valerate or dipropionate, flucinolone acetonide and hydrocortisone aceponate or butyrate, flucinolone acetonide, desonide, and hydrocortisone acetate.

14. The nail varnish as claimed in claim 13, wherein the corticoid is clobetasol or one of its pharmaceutically acceptable salts.
